(19) United States
(12) Patent Application Publication
(10) Pub. No.: US 2011/0008368 A1
(43) Pub. Date: Jan. 13, 2011

(54) METHODS OF MODULATING THE OX40 RECEPTOR TO TREAT CANCER

(75) Inventors: Yong-Jun Liu, Pearland, TX (US); Tomoki Ito, Hirakata-city (JP)

Correspondence Address: Nielsen IP Law LLC, 1177 West Loop South, Suite 1600, Houston, TX, 77027, US

(73) Assignee: BOARD OF REGENTS, THE UNIVERSITY OF TEXAS SYSTEM, Austin, TX (US)

(21) Appl. No.: 12/861,135

(22) Filed: Aug. 23, 2010

Related U.S. Application Data
(63) Continuation-in-part of application No. 11/659,266, filed on Jun. 4, 2008, now abandoned.
(60) Provisional application No. 60/759,217, filed on Jan. 13, 2006.

(51) Int. Cl. A61K 39/395; A61P 35/00; A61P 37/04; C40B 30/04

(52) U.S. Cl. 424/172.1; 506/9

(57) ABSTRACT

Numerous disease states, such as human allergic, autoimmune, and autoimmune diseases, and cancer, may be treated by targeting OX40/OX4OL. OX4OL inhibits the generation of Tr1 cells from naïve and memory CD4+ T cells. This unique function of OX4OL is not shared by two other costimulatory TNF-family members, GITR-ligand and 4-1BB-ligand. It has been shown that signaling the OX40-receptor on human T cells by antibodies, small molecules, or the OX4OL modulates the generation and function of IL-10 producing Foxp3⁺ Treg immunosuppressive T cells and blocks Foxp3⁺ Treg function. Further, provided are high throughput methods for identifying compounds that can inhibit the immunosuppressive function of IL-10 producing Tr1 cells.

### CROSS-REFERENCE TO RELATED APPLICATION

This patent application is a continuation-in-part of U.S. patent application Ser. No. 11/659,266 filed Jun. 4, 2008 which is the US National Stage Entry of International Application No. PCT/US07/01228 filed Jan. 16, 2007 which claims priority to U.S. Provisional patent application Ser. No. 60/759,217, filed Jan. 13, 2006, which is incorporated herein by reference.

### FIELD OF INVENTION

This invention relates generally to modulation of the OX40-receptor activation, and more particularly, to modulating the OX40-receptor to inhibit the immunosuppressive function of Interleukin (IL)-10-producing CD4⁺ type 1 regulatory T cells (“Tr1 cells”) and Foxp3⁺-expressing regulatory T cells (also sometimes referred to herein as “Foxp3⁺ T-reg” cells), and the generation of Tr1 cells from CD4⁺ cells or naïve cells and IL-10 production.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### BACKGROUND OF THE INVENTION

Tr1 cells have a critical role in peripheral tolerance. Tr1 cells are particularly important in limiting tissue damage to the host during inflammatory immune responses. The generation of Tr1 cells accompanies both TH1 and TH2 immune responses in vivo and in vitro.

Tr1 cells are generated from naïve CD4⁺ T cells during an antigen-driven T cell immune response. Tr1 cells are anergic in response to signaling through TCR, CD28 and IL-2 receptors and have the ability to suppress antigen-driven proliferation of naïve CD4⁺ T cells in vivo and in vitro. Tr1 cells have the ability to inhibit the development of autoimmune diseases and limit the magnitude of immune responses to microbial pathogens.

While the molecular signals that lead to the Tr1 cells have been studied, little is known about the molecular signals that negatively regulate the generation of these cells. Although immunosuppressive drugs, cytokines, costimulatory molecules, and DCs have been implicated in the induction of Tr1 cells, signals that negatively regulate the generation of Tr1 cells remain elusive.

### BRIEF SUMMARY OF THE INVENTION

Activation of the OX40 receptor blocks Tr1 generation from naïve or memory CD4⁺ T cells as well as IL-10 production from Tr1 cells and the immunosuppressive function of the Tr1 cells. Activation of the OX40 receptor also blocks IL-10 production by Foxp3⁺ T-reg cells and immunosuppressive function. As such, methods of treating cancer are provided herein by administering to a subject in need thereof an OX40 ligand or other agonist of the OX40 receptor whereby the agonist modulates the activation of the OX40 receptor to block IL-10 cytokine secretion and/or the Tr1 and Foxp3⁺ T-reg cells overall immunosuppressive function. Also, provided herein are methods of inhibiting the generation of naïve or memory CD4+ T cells by administering to a subject in need thereof an agonist or anti-OX40 monoclonal antibody agonist. Here, the monoclonal antibody essentially mimics the OX40 ligand and triggers the OX40 receptor on Tr1 and/or on natural T regulatory cells (“nTregs”), the “Foxp3⁺ T-regs.” Such anti-OX40 monoclonal antibody agonist may also block the IL cytokine secretion and the immunosuppressive function of this cell.

As we show and describe herein, OX40L inhibits the generation and function of IL-10-producing Tr1 cells from naïve and memory CD4+ T cells that were induced by the immunosuppressive drugs dexamethasone and vitamin D3. We have discovered that OX40L inhibits the generation and function of IL-10 producing regulatory T cells. These discoveries demonstrate that signaling OX40 by OX40L suppresses the generation of human IL-10 producing immunosuppressive T cells in culture. This unique function of OX40L is not shared by two other costimulatory TNF-family members, GITR-ligand and 4-1BB-ligand. OX40L also strongly inhibits the generation and function of IL-10-producing Tr1 cells induced by two physiological stimuli provided by inducible costimulatory ligand and immature DCs. Signaling the OX40 receptor on human T cells by monoclonal antibodies, small molecules, or by the OX40L, or protein having at least 90 percent homology thereto, modulates and regulates the generation and function of IL-10 producing immunosuppressive T cells.

The discovery lends to numerous applications of treatment. For example, agonistic antibodies, small molecules, or OX40L could be used to suppress the generation and the function of IL-10 producing immunosuppressive T cells and therefore could be used to enhance immune responses to treat cancer and infectious diseases, or as an adjuvant for cancer vaccines. Antagonistic antibodies to OX40 or to OX40L, or antagonistic small molecules, could be used to enhance the generation and the function of IL-10-producing immunosuppressive T cells and therefore could be used for the development of therapies for autoimmune diseases and graft versus host diseases. Our discovery also provides for high throughput methods for screening antibodies or small molecules either activating the OX40 receptor (or conversely blocking OX40 signaling) on T cells for the development of therapeutics for cancer, or alternatively, autoimmune diseases, and graft versus host diseases.

### BRIEF DESCRIPTION OF THE DRAWINGS

Some of the features and benefits of the present invention having been stated, others will become apparent as the description proceeds when taken in conjunction with the accompanying drawings, in which:

FIG. 1A is a plurality of graphs of the intracellular analysis of cytokine production by naïve CD4⁺ T cells by flow cytometry.

FIG. 1B is a plurality of graphs of cytokine production by naïve CD4⁺ T cells by ELISA.

FIG. 1C is a graph of suppressive function in T cells by [³H]thymidine incorporation.

FIG. 2A is a plurality of graphs of the intracellular analysis of cytokine production by memory CD4⁺ T cells by flow cytometry.

FIG. 2B is a graph of IL-10 production by memory CD4⁺ T cells by ELISA.

FIG. 3A is a plurality of graphs of the intracellular analysis of cytokine production by naïve CD4⁺ T cells by flow cytometry.

FIG. 3B is a graph of IL-10 production by naïve CD4⁺ T cells by ELISA.

FIG. 3C is a graph of the number of viable T cells counted.

FIG. 4A is a plurality of graphs of the intracellular analysis of cytokine production by naïve CD4⁺ T cells by flow cytometry.

FIG. 4B is a graph of IL-10 production by naïve CD4⁺ T cells by ELISA.

FIG. 4C is a plurality of graphs of the intracellular analysis of cytokine production by memory CD4⁺ T cells by flow cytometry.

FIG. 4D is a graph of IL-10 production by memory CD4⁺ T cells by ELISA.

FIG. 4E is a plurality of graphs of the intracellular analysis of cytokine production by naïve CD4⁺ T cells by flow cytometry.

FIG. 4F is a plurality of graphs of IL-10 production by naïve CD4⁺ T cells by ELISA.

FIG. 5 is a plurality of graphs of IL-10 production by regulatory T cells by ELISA.

FIGS. 6A, 6B, 6C and 6D show that OX40L (OX40 ligand) expressing L cells enhance effector T-cells proliferation and block the proliferation of Foxp3⁺ T-regs (also referred to herein as a “nTreg” cell) immunosuppressive function in lymphoma cells.

FIGS. 7A and 7B show the decreasing tumor volume in mice bearing A20 murine lymphoma tumors when an anti-OX40 antibody agonist is administered.

### DETAILED DESCRIPTION

The present invention now will be described more fully hereinafter with reference to the accompanying drawings in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

OX40/OX40-ligand (OX40 Receptor)/(OX40L) are a pair of costimulatory molecules critical for T cell proliferation, survival, cytokine production, and memory cell generation. Early in vitro experiments demonstrated that signaling through OX40 on CD4⁺ T cells lead to TH2, but not TH1 development. These results were supported by in vivo studies showing that blocking OX40/OX40L interaction prevented the induction and maintenance of TH2-mediated allergic immune responses. However, blocking OX40/OX40L interaction ameliorates or prevents TH1-mediated diseases. Furthermore, administration of soluble OX40L or gene transfer of OX40L into tumors were shown to strongly enhance anti-tumor immunity in mice. Recent studies also suggest that OX40/OX40L may play a role in promoting CD8 T cell-mediated immune responses. As discussed herein, OX40 signaling blocks the inhibitory function of CD4⁺CD25⁺ naturally occurring regulatory T cells and the OX40/OX40L pair plays a critical role in the global regulation of peripheral immunity versus tolerance.

We discovered that function of OX40L is the negative regulation of the generation of IL-10-producing Tr1 cells induced by immunosuppressive agents Dex and Vit D, ICOSL, or immature DCs. This discovery demonstrates a general mechanism by which OX40L enhances immunity and breaks immunological tolerance.

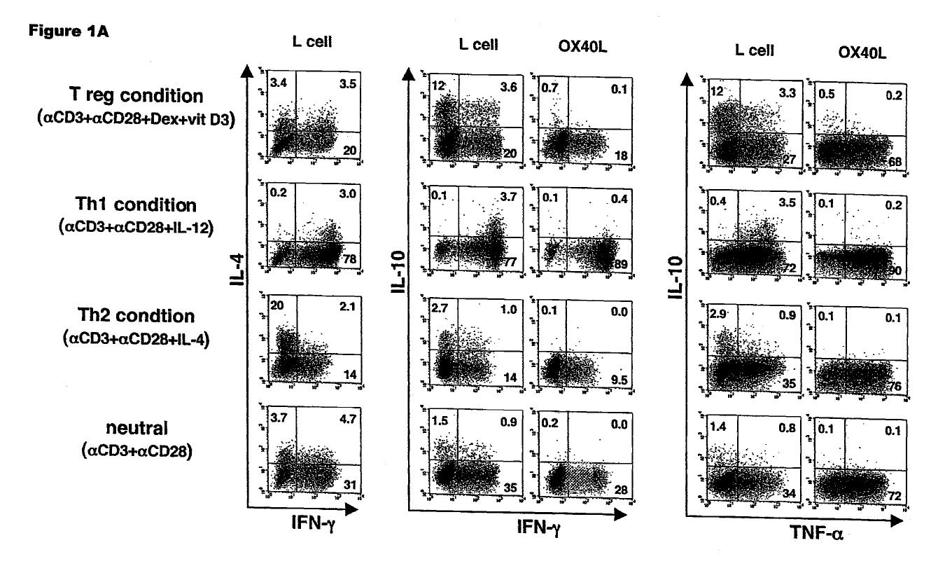

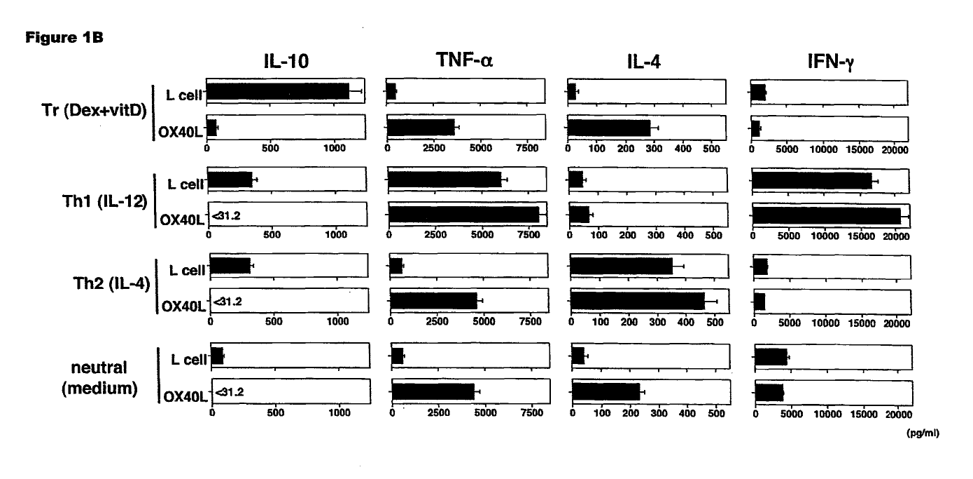

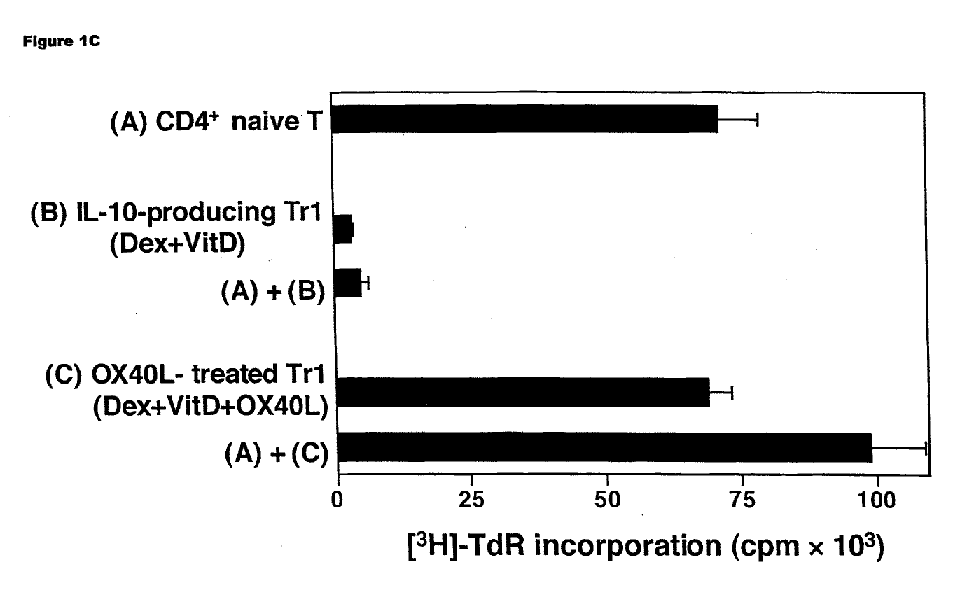

We have discovered that OX40L inhibits the generation of IL-10-producing Tr1 cells from CD4⁺ T cells induced by Dexamethasone and vitamin D3. It is known that a combination of the immunosuppressive drugs Dex and Vit D3 consistently induce the differentiation of naïve CD4⁺ T cells into IL-10-producing Tr1 cells. To investigate whether OX40L can inhibit the generation and function of IL-10-producing Tr1 cells, naïve CD4⁺ T cells were cultured with anti-CD3 plus anti-CD28 mAbs in the presence or absence of OX40L-transfected L cells in four different culture conditions including: 1) Tr1 (Dex and vit D3); 2) TH1 (IL-12); 3) TH2 (IL-4); or 4) neutral (medium alone) for 7 days (FIG. 1A). IL-10 production by the primed T cells was analyzed by intracellular cytokine staining and ELISA.

In the experiments of FIG. 1A, an intracellular analysis of cytokine production by naïve CD4⁺ T cells was conducted by flow cytometry. Naïve CD4⁺ T cells were cultured with anti-CD3 and anti-CD28 mAbs in the presence of IL-2 on parental L cells or OX40L-L cells with the indicated recombinant cytokines or reagents for 7 days. Percentages of the respective cytokine-producing T cells are indicated in each dot blot profile. The results show that OX40L inhibits the generation of IL-10-producing Tr1 cells from naïve CD4⁺ T cells induced by the different polarizing signals. As shown in FIG. 1A, between 2% to 4% of IL-10-producing Tr1 cells were generated from naïve CD4⁺ T cells cultured in neutral or TH1 or TH2 conditions. More than 15% of IL-10-producing Tr1 cells were generated in culture with Dex plus vit D3. The addition of OX40L completely blocked the generation of IL-10-producing Tr 1 cells, while promoting the generation of TNF-α-producing T cells in all culture conditions.

These data were confirmed by ELISA data (FIG. 1B). In the experiments of FIG. 1B, cytokine production by naïve CD4⁺ cells was measured in supernatants after restimulation with anti-CD3 and anti CD28 mAbs for 24 h by ELISA. Naïve CD4⁺ T cells were cultured with anti-CD3 and anti-CD28 mAbs in the presence of IL-2 on parental L cells or OX40L-L cells with the indicated recombinant cytokines or reagents for 7 days. The data are shown as mean ±SEM of four independent experiments. The results show that OX40L inhibits the generation of IL-10-producing Tr1 cells from naïve CD4⁺ T cells induced by the different polarizing signals.

Naïve CD4⁺ T cells primed with Tr1 condition (Dex plus vit D3) were anergic and had the ability to suppress the proliferation of naïve CD4⁺ T cells in response to anti-CD3 plus anti-CD28 mAbs (FIG. 1C). In the experiments of FIG. 1C, suppressive function in T cells was measured by [³H]thymidine incorporation. Mixtures of the indicated T cell populations were restimulated by anti-CD3 and anti-CD28 mAbs. Error bars represent SEM of triplicate wells. It was discovered that naïve CD4⁺ T cells primed with the same Tr1 condition in the presence of OX40L proliferated vigorously and failed to inhibit the proliferation of naïve CD4⁺ T cells in response to anti-CD3 plus anti-CD28 mAbs. As understood by those of skill in the art, these data suggest that OX40L blocks the generation of functional Tr1 cells from naïve CD4⁺ T cells induced by Dex and Vit D3.

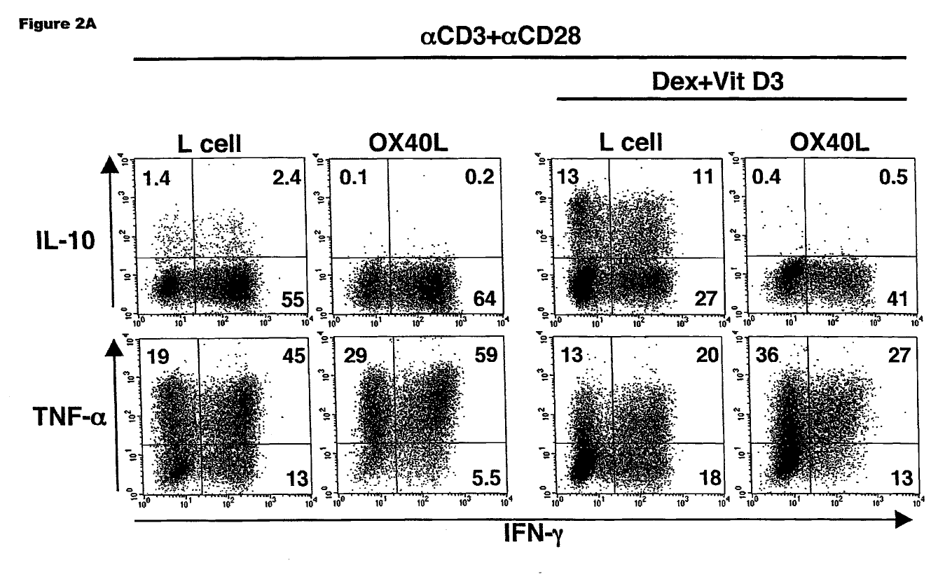

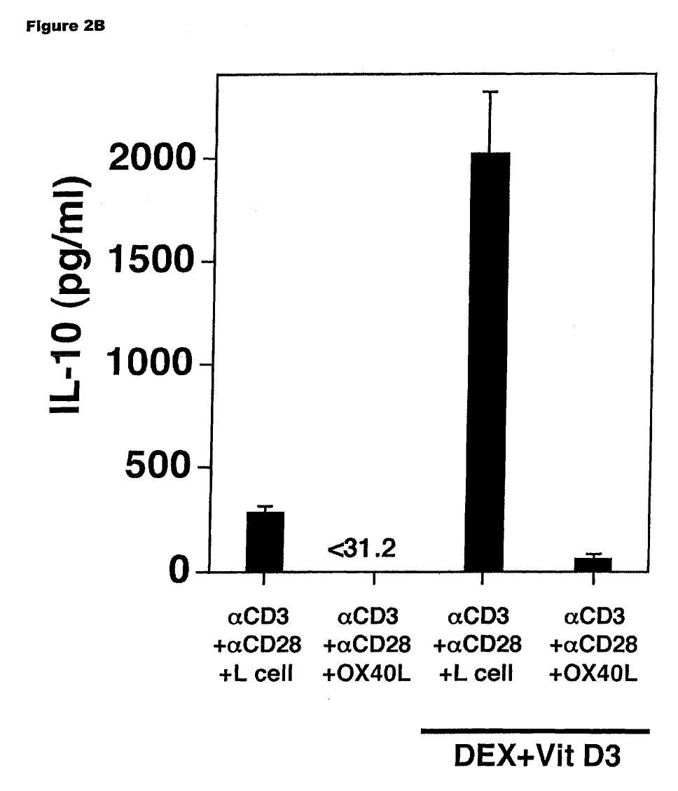

Also investigated was whether IL-10-producing Tr1 cells can be generated from memory CD4⁺CD45RA⁻CD45RO⁺ T cells, and whether OX40L can inhibit the generation of IL-10-producing Tr1 cells from memory CD4⁺ T cells. Memory CD4⁺CD45RA⁻CD45RO⁺ T cells were cultured for 7 days with anti-CD3 plus anti-CD28 mAbs in the presence or absence of OX40L-transfected L cells Tr1 condition (Dex plus vit D3). In the experiments of FIG. 2A, an intracellular analysis of cytokine production by CD4⁺ memory T cells was conducted by flow cytometry. Memory CD4⁺CD45RO⁺CD25⁻ memory T cells were cultured with anti-CD3, anti-CD28 mAbs, and IL-2 on parental L cells or OX40L-L cells in the presence or absence of Dex plus vit D3 for 7 days. Percentages of the respective cytokine-producing T cells are indicated in each dot blot profile. The results show that OX40L inhibits the generation of IL-10-producing Tr1 cells from memory CD4⁺ T cells under a condition with Dex plus Vit D3. FIG. 2A shows that large numbers of IL-10-producing cells (>20%) were generated from CD4⁺ memory T cells in culture with Dex plus vit D3. The addition of OX40L completely blocked the generation of IL-10-producing Tr1 cells and promoted generation of TNF-α-producing cells from memory CD4⁺ T cells.

The ability of Dex plus vit D3 to promote IL-10 production from memory CD4⁺ T cells, and that this ability can be inhibited by OX40L, were confirmed by IL-10 ELISA analyses (FIG. 2B). In the experiments of FIG. 2B, IL-10 production by memory CD4⁺ T cells was measured in supernatants after restimulation with anti-CD3 and anti-CD28 mAbs for 24 h by ELISA. The data are shown as mean ±SEM of four independent experiments. The results show that OX40L inhibits the generation of IL-10-producing Tr1 cells from memory CD4⁺ T cells under a condition with Dex plus Vit D3.

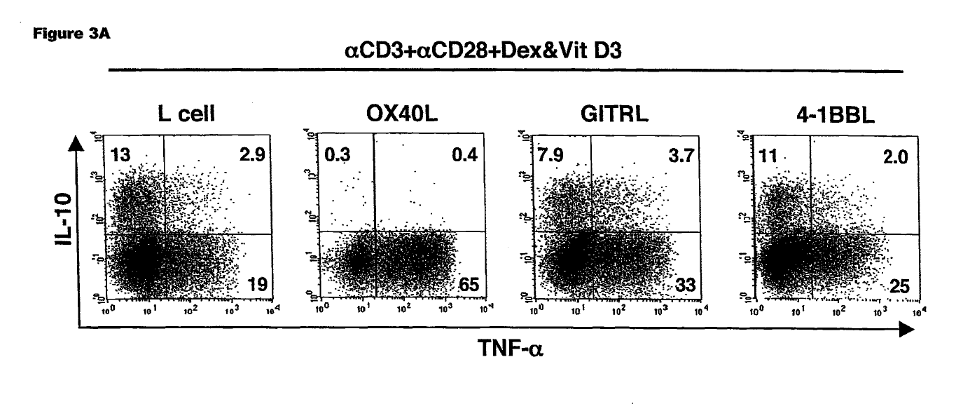

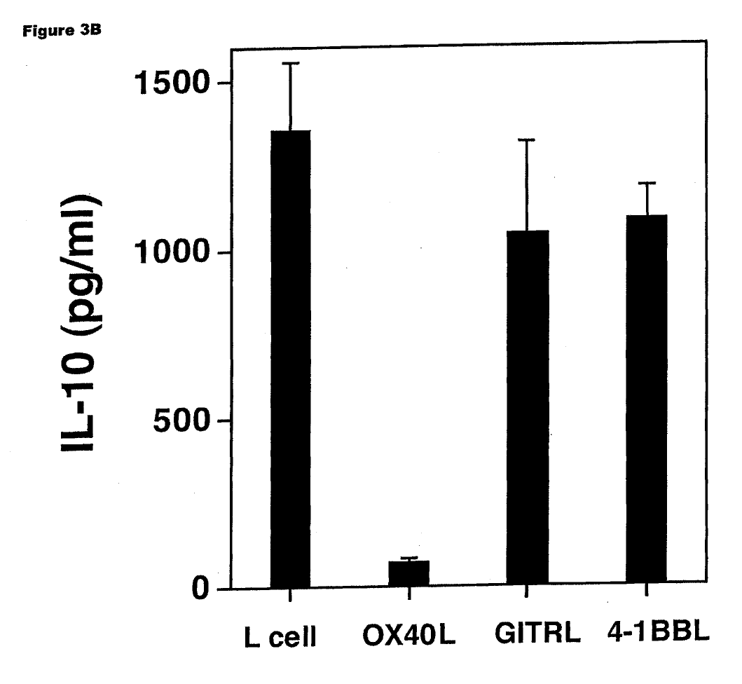

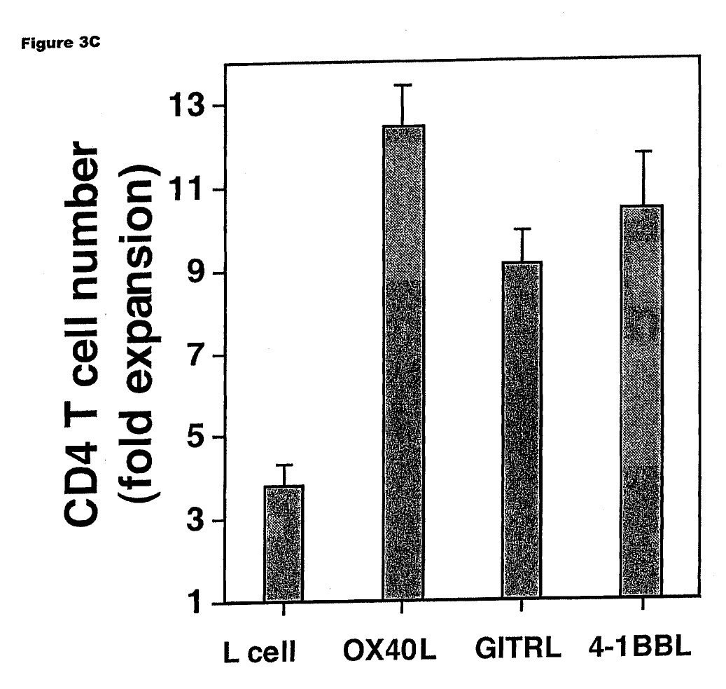

It was further discovered that OX40L inhibits the generation of IL-10-producing Tr1 cells, while other TNF-family members (GITRL and 4-1BBL) do not. Within the TNF-superfamily, OX40L, glucocorticoid-induced TNF receptor-ligand (GITRL), and 4-1BB-ligand (4-1BBL) have costimulatory function for T cells. To investigate whether OX40L was unique in the inhibition of IL-10-producing Tr1 cells, naïve CD4⁺ T cells were cultured with anti-CD3 plus anti-CD28 mAbs with Dex plus vit D3, with parental L cells or L cells transfected with OX40L, GITRL, or 4-1BBL for 7 days. While OX40L, GITRL, and 4-1BBL all promoted the generation of TNF-α-producing cells, only OX40L inhibited the generation of IL-10-producing Tr 1 cells (FIGS. 3A and B).

In the experiments of FIG. 3A, an intracellular analysis of cytokine production by naïve CD4⁺ T cells was conducted by flow cytometry. Naïve CD4⁺ T cells were cultured with anti-CD3, anti-CD28 mAbs, and IL-2 on parental L cells, OX40L-L cells, GITRL-L cells, or 4-1BBL-L cells in the presence of Dex plus vit D3 for 7 days. Percentages of the respective cytokine-producing T cells are indicated in each dot blot profile. The results show that OX40L but not GITRL nor 4-1BBL inhibits the generation of IL-10-producing Tr1 cells.

In the experiments of FIG. 3B, IL-10 by naïve CD4⁺ cells was measured in supernatants after restimulation with anti-CD3 and anti CD28 mAbs for 24 h by ELISA. The data are shown as mean ±SEM of four independent experiments. The results show that OX40L but not GITRL nor 4-1BBL inhibits the generation of IL-10-producing Tr1 cells.

OX40L, GITRL, and 4-1BBL all promoted the expansion of total T cell numbers (FIG. 3C). In the experiments of FIG. 3C, the number of viable T cells was counted. The data are shown as mean ±SEM of four independent experiments.

As understood by those of skill in the art, the results of FIG. 3A-C show that OX40L, but not GITRL nor 4-1BBL, inhibits the generation of IL-10-producing Tr1 cells. These data suggest that among the three members of TNF-superfamily known to costimulate T cells, OX40L has a novel and unique function in inhibiting the generation of IL-10-producing Tr1 cells.

It was further discovered that OX40L inhibits the generation of IL-10-producing Tr1 cells induced by ICOSL or immature DCs. ICOS and CD28 represent the two positive costimulatory receptors within the CD28 family expressed on T cells. Signaling through ICOS by agonistic Abs or ICOSL has been shown to promote CD4⁺ T cells to produce IL-10. To investigate whether OX40L can inhibit the ability of ICOS to induce IL-10 production by CD4⁺ T cells, naïve and memory CD4⁺ T cells were cultured with anti-CD3 in the presence of ICOSL-transfected L cells, or ICOSL-transfected L cells in the presence of OX40L for 7 days.

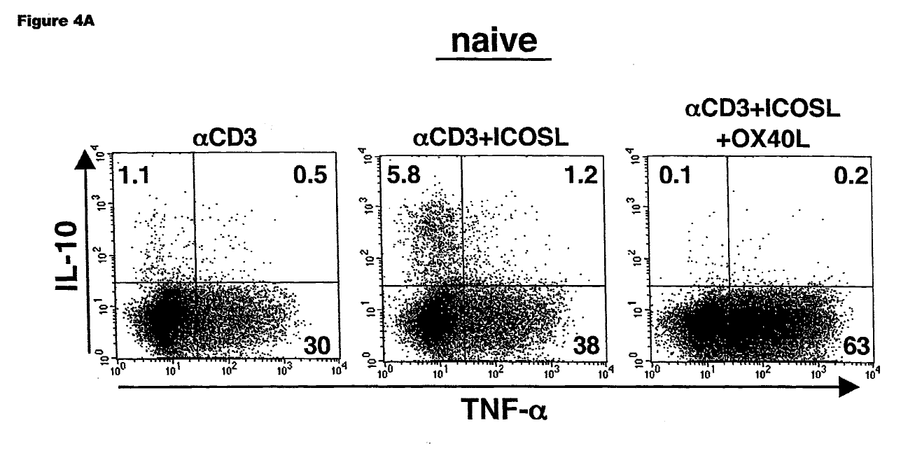

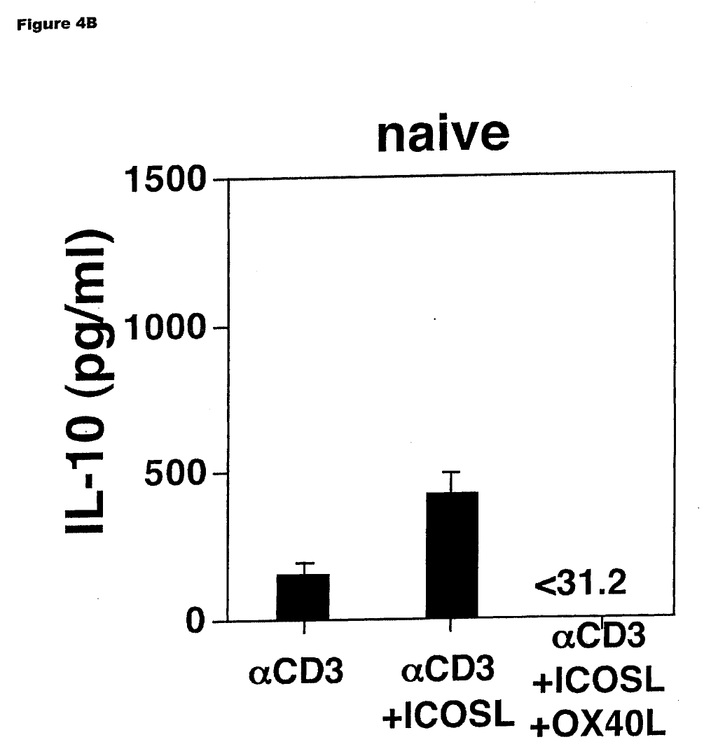

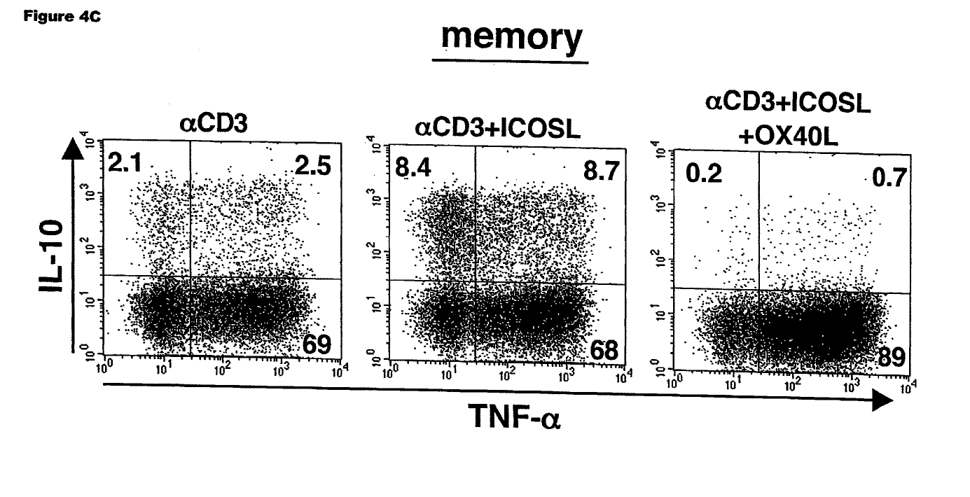

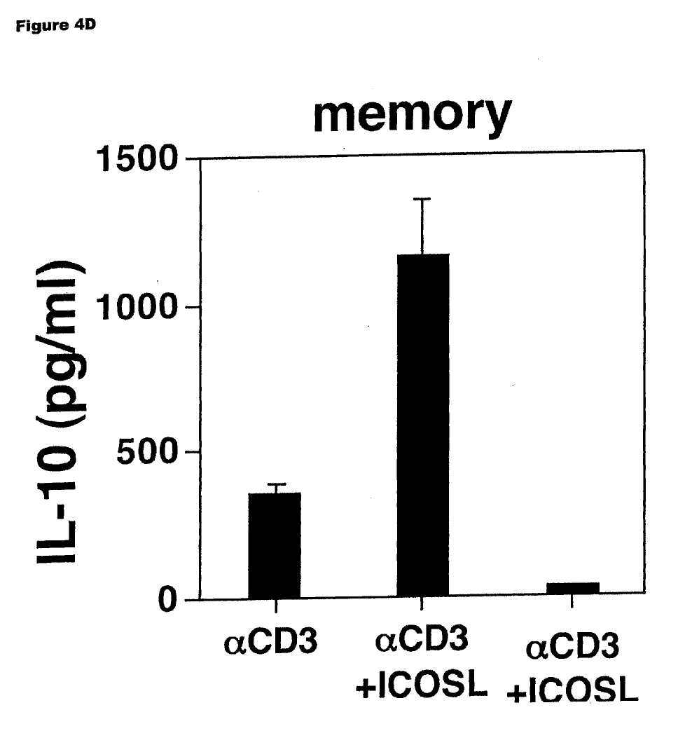

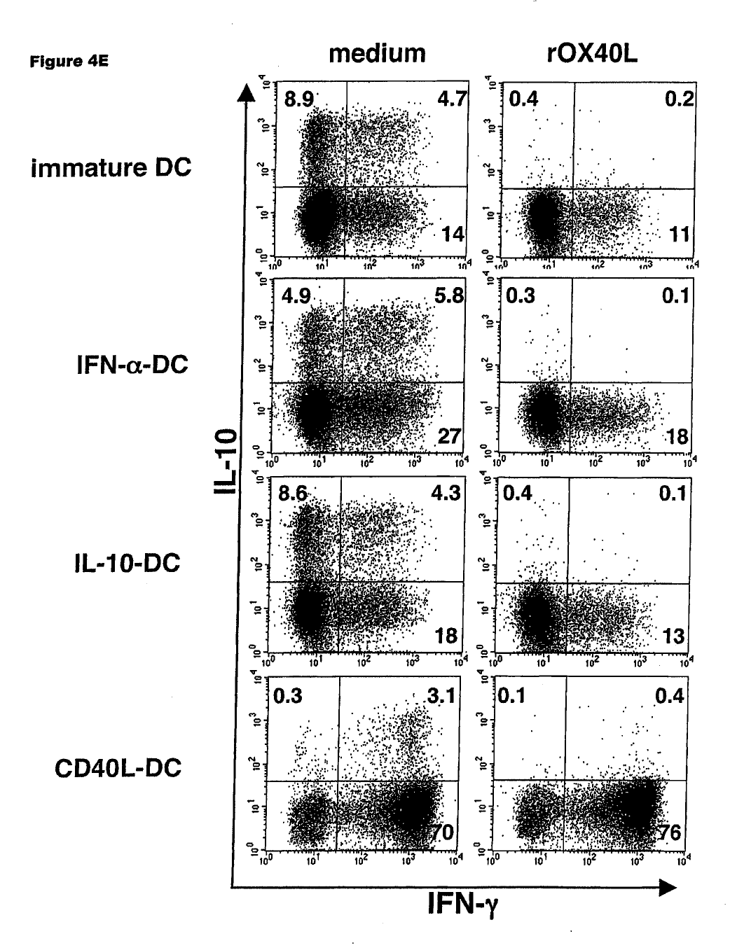

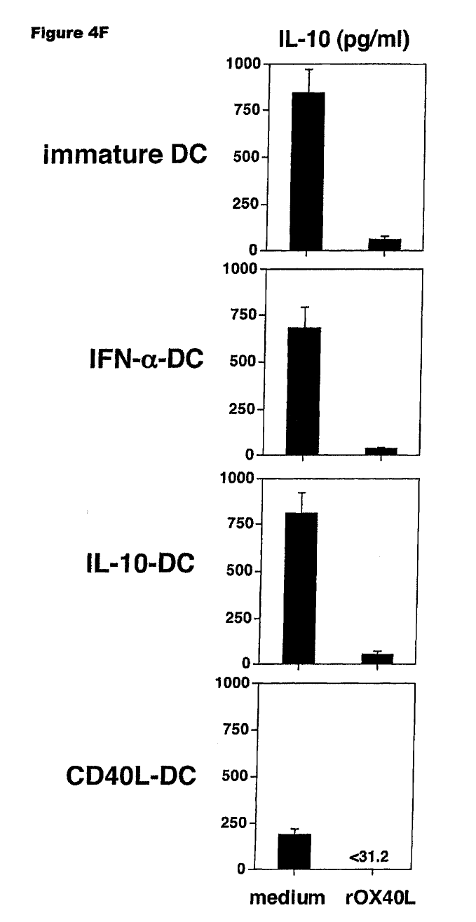

In the experiments of FIG. 4A, an intracellular analysis of cytokine production by naïve CD4⁺ T cells was conducted by flow cytometry. Naïve CD4⁺ T cells were cultured for 7 days on parental L cells, on a mixture of ICOSL-L cells and L cells, or on a mixture of ICOSL-L cells and OX40L-L cells, which were pre-coated with anti-CD3 mAb. Percentages of the respective cytokine-producing T cells are indicated in each dot blot profile. The results show that OX40L inhibits the generation of IL-10-producing Tr1 cells from naïve CD4⁺ T cells induced by ICOSL.

In the experiments of FIG. 4B, IL-10 production by naïve CD4⁺ cells was measured in supernatants after restimulation with anti-CD3 and anti-CD28 mAbs for 24 h by ELISA. Naïve CD4⁺ T cells were cultured for 7 days on parental L cells, on a mixture of ICOSL-L cells and L cells, or on a mixture of ICOSL-L cells and OX40L-L cells, which were pre-coated with anti-CD3 mAb. The data are shown as mean ±SEM of three independent experiments. The results show that OX40L inhibits the generation of IL-10-producing Tr1 cells from naïve CD4⁺ T cells induced by ICOSL.

In the experiments of FIG. 4C, an intracellular analysis of cytokine production by memory CD4⁺ T cells was conducted by flow cytometry. Memory CD4⁺ T cells were cultured for 7 days on parental L cells, on a mixture of ICOSL-L cells and L cells, or on a mixture of ICOSL-L cells and OX40L-L cells, which were pre-coated with anti-CD3 mAb. Percentages of the respective cytokine-producing T cells are indicated in each dot blot profile. The results show that OX40L inhibits the generation of IL-10-producing Tr1 cells from memory CD4⁺ T cells induced by ICOSL.

In the experiments of FIG. 4D, IL-10 production by memory CD4⁺ T cells was measured in supernatants after restimulation with anti-CD3 and anti-CD28 mAbs for 24 h by ELISA. Memory CD4⁺ T cells were cultured for 7 days on parental L cells, on a mixture of ICOSL-L cells and L cells, or on a mixture of ICOSL-L cells and OX40L-L cells, which were pre-coated with anti-CD3 mAb. The data are shown as mean ±SEM of three independent experiments. The results show that OX40L inhibits the generation of IL-10-producing Tr1 cells from memory CD4⁺ T cells induced by ICOSL.

The results of the experiments of FIGS. 4A-D show that ICOSL significantly promoted the generation of IL-10-producing cells from both naïve and memory CD4⁺ T cells. The addition of OX40L completely inhibited the generation of IL-10-producing cells from both naïve and memory CD4⁺ T cells, while strongly promoting the generation of cells producing TNF-α.

It is known that immature DCs or DCs treated with IFN-α or IL-10 can induce naïve CD4⁺ T cells to differentiate into IL-10-producing Tr1 cells. It was investigated whether OX40L could inhibit the generation of IL-10-producing Tr1 cells induced by DCs. As shown in FIG. 4E, immature DCs or DCs treated with IL-10 or IFN-α all induced the generation of more than 10% of IL-10-producing Tr1 cells from naïve CD4⁺ T cells. By contrast, DCs activated by CD40L induce a strong TH1 response, accompanied by the generation of about 3% IL-10-producing Tr1 cells. Addition of recombinant OX40L in DC-T cell cultures completely inhibited the generation of IL-10-producing Tr1 cells induced by immature DCs and DCs treated with IL-10 and IFN-α. In addition, OX40L also inhibited the generation of the residual number of IL-10-producing Tr1 cells induced by the CD40L activated mature DCs. In the experiments of FIG. 4E, an intracellular analysis of cytokine production by CD4⁺ naïve T cells was conducted by flow cytometry. Naïve CD4⁺ T cells were cocultured in the presence or absence of soluble recombinant OX40L for 7 days with immature DCs or DCs cultured with IFN-α, IL-10, and CD40L. Percentages of the respective cytokine-producing T cells are indicated in each dot blot profile. The results show that OX40L inhibits the generation of IL-10-producing Tr1 cells from CD4⁺ T cells induced by DCs

The ability of OX40L to inhibit the generation of IL-10-producing Tr1 cells induced by DCs was confirmed by ELISA data (FIG. 4F). In the experiments of FIG. 4F, IL-10 production by naïve CD4⁺ cells was measured in supernatants after restimulation with anti-CD3 and anti-CD28 mAbs for 24 h by ELISA. Naïve CD4⁺ T cells were cocultured in the presence or absence of soluble recombinant OX40L for 7 days with immature DCs or DCs cultured with IFN-α, IL-10, and CD40L. The data are shown as mean ±SEM of three independent experiments. The results show that OX40L inhibits the generation of IL-10-producing Tr1 cells from CD4⁺ T cells induced by DCs. Thus, these data demonstrate that OX40L could inhibit the generation of IL-10-producing Tr1 cells induced by more physiological signals provided by ICOSL and DCs.

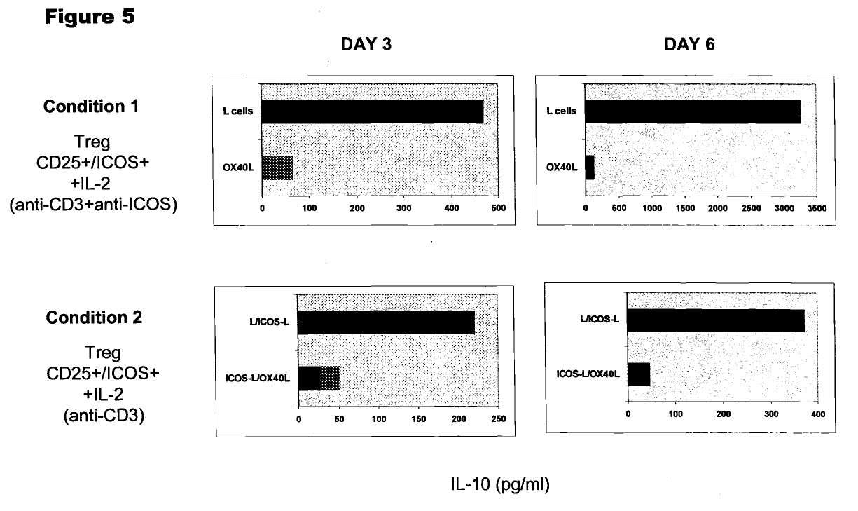

It has been previously suggested that regulatory T cells are highly represented in the area of B cell non-Hodgkin's lymphoma and that B cells are involved in the recruitment of regulatory T cells into the area of the lymphoma. It was investigated whether influencing the signaling of OX40-receptors, such as by OX40L, could provide a therapy against B cell lymphoma. Frozen samples from B cell lymphoma patients were used to estimate the ability of OX40L to shut down IL-10 producing regulatory T cells. The samples used were follicular lymphoma obtained from a spleen specimen prior to any treatment. The cells were thawed, with 400×10⁶ frozen cells yielding 127×10⁶ live cells and 33.9×10⁶ dead cells (79% viability). A sufficient number of CD25+ cells were identified by FACS staining. In the experiments of FIG. 5, IL-10 production by regulatory T cells was determined by ELISA. Regulatory T cells (Treg cells) were cultured under two different conditions. In condition 1, CD25+/ICOS+ cells were cultured with anti-CD3 in the presence of IL-2 (900 μl/ml) on parental L cells or OX40L-L cells with anti-ICOS antibody for 3-6 days. In condition 2, CD25+/ICOS+ cells were cultured with anti-CD3 in the presence of IL-2 (900 μl/m1) on ICOS-L-L cells or a mixture of OX40L-L can ICOS-L-L cells for 3 to 6 days. Cytokine production was measured in the supernatants by ELISA. The results show that OX40L greatly inhibited IL-10 production by Treg cells.

The present findings, that OX40L has the capacity to inhibit the generation and function of IL-10-producing Tr1 cells induced by the immunosuppressive drugs Dex plus vit D3, ICOSL, or DCs, highlights a novel mechanism by which OX40L promotes immunity and breaks tolerance during different forms of CD4- or CD8-mediated immune responses, as would be understood by one of skill in the art. The ability of OX40L to inhibit the generation of IL-10-producing Tr1 cells during both IL-12 induced TH1 or IL-4 induced TH2 responses suggest that OX40L may control the magnitude of TH1- or TH2-mediated immune responses. Furthermore, the ability of OX40L to inhibit the generation of IL-10-producing Tr1 cells appears to be a unique property of OX40L, because the two other TNF-family members GITRL and 4-1BBL do not have this functional property. Moreover, the ability of OX40L to inhibit IL-10 production by Treg cells identifies OX40L as a potent treatment for B cell lymphoma and other cancers.

Many molecules have been identified that promote the generation of IL-10-producing Tr1 cells, including IL-10, IFN-α, ICOSL, and immunosuppressive compounds such as Dex plus vit D3. OX40L represents a potent inhibitor for the generation of IL-10-producing Tr1 cells not only from naïve CD4⁺ T cells, but also from memory CD4⁺ T cells and regulatory T cells. This novel property of OX40/OX40L may explain a recent report showing that OX40 signaling allows anergic autoreactive T cells to acquire effector cell functions. Targeting OX40/OX40L thus provides for treatments for human allergic and autoimmune diseases and as well as for the development of treatments for human infectious diseases and cancer.

T regulatory cells are a component of the immune system that suppresses the immune responses of other cells. This is an important “self-check” built into the immune system to prevent excessive reactions. Regulatory T cells come in many forms, including those that express the CD8 transmembrane glycoprotein (CD8+ T cells); those that express CD4, CD25 and Foxp3⁺. Foxp3⁺ is sometimes also referred to as: CD4+CD25+ regulatory T cells; CD4⁺Foxp3⁺ regulatory T cells; and/or “nTregs” Foxp3⁺ T-regs are involved in shutting down immune responses after they have successfully tackled invading organisms, and also in regulating immune responses that may potentially attack one's own tissues such as in the case of autoimmunity.

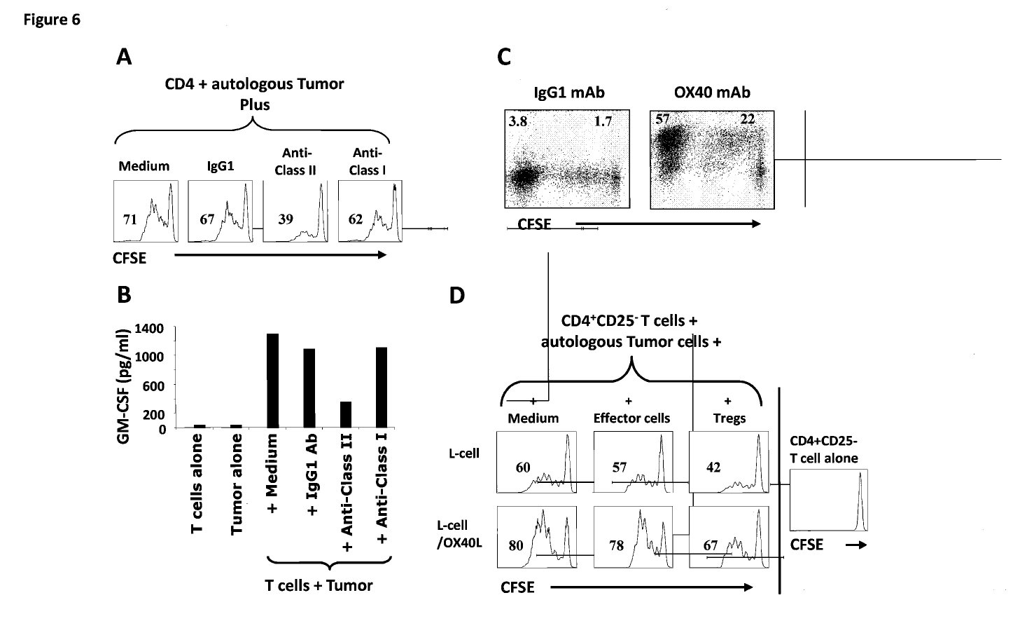

As shown in FIG. 6A, the lack of proliferation of CD4⁺ effector T cells mediated by Class II molecules is shown when tumor cells are treated with an anti-Class II antibody (an antibody that blocks the antigen presenting cell from binding to its ligand). Also, FIG. 6B shows that tumor reactive T cells do not secret GM-CSF when the anti-Class II antibody is administered to T cells and tumor. FIG. 6C shows that CD4⁺ T cells express the OX40 receptor. FIG. 6D shows that the immunosuppressive function of nTregs is blocked by OX40L-L cells. Therefore, the OX40L cells (expressing the OX40L) block immunosuppressive function of the Treg cells.

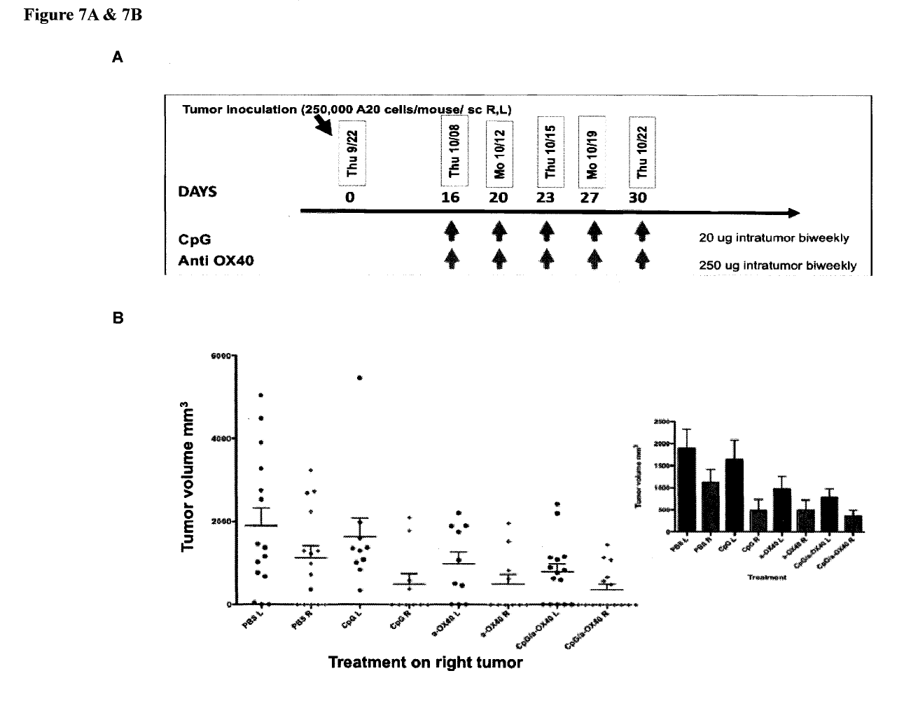

FIGS. 7A and 7B are the results of in vivo experiments proving that the treatment of a murine lymphoma tumor with an agonist anti mouse anti OX40 commercial monoclonal antibody (clone OX86) display antitumor activity per se and in combination with a Toll like receptor 9 ligand (CpGB). These results are proof that the use of an agonist anti human OX40 antibody in human therapy may be effective as well, alone or in combination with other adjuvants.

Specifically, as a proof of principle we explored the anti tumor efficacy of the therapeutic use of a combination of an agonist anti mouse OX40 antibody and a TLR9 ligand. For this purpose we used a murine lymphoma model. FIG. 7A shows that the intratumor vaccination using the TLR9 ligand (CpGB) in combination with the anti-murine OX40 (OX86) antibody, induces antitumor response on a subcutaneous murine lymphoma model. Tumor cells were inoculated simultaneously on the right and left flank but the therapeutic vaccination was performed only on the right tumor. The left tumor showed also size reduction compared with the PBS control, suggesting systemic antitumor effect of the combinatorial therapy. These in vivo data provide further support to develop agonist anti human anti-OX40 monoclonal antibodies into novel tumor therapy in humans. FIG. 7A provides the results from BalbC mice bearing established A20 murine lymphoma tumors on the right and left flanks were treated with a combination of CpGB with or without anti-murine OX40 monoclonal antibody injected on the right tumor only at the indicated time. FIG. 7B shows tumor volume in the different treated groups (Control PBS, CpG alone, anti-OX40 alone, and CpG+ anti OX40was determined at the end of the vaccination protocol on both flank tumors.

The present discoveries also provide for high throughput screening methods. More specifically, and as understood by those skilled in the art, high throughput methods to screen for antagonistic or agonistic monoclonal antibodies or small molecules that bind to OX40-receptors, and that can inhibit the generation and function of IL-10 producing cells or promote the generation and function of IL-10 producing cells, are made possible. In one such method, a human T cell line (SU-DHL-1) having the ability to produce IL-10 was transfected with the human OX40-gene (SUOX40). 100,000 SUOX40 cells were cultured with either 100,000 mouse fibroblast cells (L cells) or 100,000 mouse fibroblast cells expressing the human OX40-ligand (OX40-ligand L cells) in 96 well-plates. After 48 hours of culture, culture supernatants were collected for the measurement of IL-10 by IL-10-specific ELISA.

In a representative experiment, 100,000 SUOX40 cells produced up to 6,000 pg/ml IL-10 cultured in the absence of OX40-ligand. In the presence of OX40-ligand, 100,000 SUOX40 cells produced less than 1,000 pg/ml IL-10. This culture method may be used to screen for, inter alia, antagonistic monoclonal antibodies or small molecules that block the ability of OX40-ligand to inhibit IL-10 production by SUOX40 cells. Alternatively, this culture method may be modified by replacing OX40-ligand expressing L cells with potential agonistic monoclonal antibodies or small molecules specific to OX40 to determine, inter alia, their ability to inhibit IL-10 production by SUOX40 cells.

The following materials and methods were used:

L cell lines. Human GITRL, OX40L, 4-1BBL, ICOSL expressing L cells were generated by retroviral mediated transduction, as understood by those of skill in the art. Briefly, full-length coding sequence for human GITRL (Accession# NM_{—}005092), OX40L (Accession# NM_{—}003326), 4-1BBL (Accession# NM_{—}003811), ICOSL (Accession# NM_{—}015259) was amplified by RT-PCR with RNA prepared from HSV-1 stimulated PBMCs. Subsequently the cDNAs were cloned into an MSCV based retroviral vector pMIGW2 and the resulting plasmids were verified by restriction enzyme digestion and DNA sequencing. To produce recombinant retroviruse, each vector was co-transfected with packaging constructs pCL-gp (gag/pol) and pHCMV-VSVg (VSV glycoprotein envelop) in HEK293T cells. Two days later, the virus containing culture supernatants were harvested and used to infect CD32 L cells at moi 100. Under this condition >95% cells were productively transduced.

Generation of monocyte-derived DCs. Isolated CD14⁺ monocytes (purity >94%) were cultured in the presence of 100 ng/ml GM-CSF and 50 ng/ml IL-4 (both from R&D) for 5 days, as understood by those of skill in the art. The resulting immature DCs were washed and cultured for 24 h with IFN-α (1000 U/ml, PBL Biomedical Laboratories), IL-10 (10 ng/ml, R&D), and irradiated CD40L-transfected L cells (DC to L cell ratio, 4:1) to obtain mature DCs, as understood by those of skill in the art.

CD4⁺ T cell stimulation. Neve CD4⁺ T cells and memory CD4⁺ T cells (each purity >99%) were isolated from PBMCs using CD4⁺ T cell Isolation Kit II (Miltenyi Biotec) followed by cell sorting (CD4⁺CD45RA⁺CD45RO⁻CD25⁻ fraction as naïve T cells and CD4⁺CD45RA⁻CD45RO⁺CD25⁻ fraction as memory T cells), as understood by those of skill in the art. 4×10⁴ freshly purified allogeneic naïve CD4⁺ T cells were cocultured with immature or cultured DCs (DC to T ratio, 1:10) in the presence or absence of recombinant human OX40L (R&D, 100 ng/ml) in round-bottomed 96-well culture plates for 7 days, as understood by those of skill in the art. Purified CD4⁺ T cells were also cultured with IL-12 (10 ng/ml, R&D), IL-4 (25 ng/ml, R&D), or combination of dexamethasone (5×10⁻⁸ M, Life Technologies) and 1alpha, 25-dihydroxyvitamin D3 (10⁻⁷ M) for 7 days in the presence of soluble anti-CD28 mAb (CD28.2, 1 μg/ml) and IL-2 (50 U/ml, R&D) on the irradiated CD32/OX40L-L cells, CD32/GITRL-L cells, CD32/4-1BBL-L cells, or parental CD32-L cells which had been pre-coated with anti-CD3 mAb (OKT3, 0.2 μg/ml) in 48-well culture plates (T cell to L cell ratio, 2.5:1), as understood by those of skill in the art. In some experiments, CD4⁺ T cells were cultured for 7 days on the CD32-L cells, mixture of CD32-L cells and CD32/ICOSL-L cells (ratio 1:1), or mixture of CD32/ICOSL-L cells and CD32/OX40L-L cells (ratio 1:1) pre-coated with anti-CD3 mAb (0.2 μg/ml) in 48-well culture plates, as understood by those of skill in the art. RPMI 1640 was used and supplemented with 10% FCS, 2 mM L-glutamine, 1 mM sodium pyruvate, penicillin G, and streptomycin for the cultures, as understood by those of skill in the art.

Analyses of T cell cytokine production. The cultured T cells were collected and washed, and then restimulated with plate-bound anti-CD3 (5 μg/ml) and soluble anti-CD28 (2 μg/ml) at a concentration of 1×10⁶ cells/ml for 24 h, as understood by those of skill in the art. The levels of IL-4, IL-10, TNF-α, and IFN-γ in the supernatants were measured by ELISA (all kits from R&D), as understood by those of skill in the art. For intracellular cytokine production, the cultured T cells were restimulated with 50 ng/ml of PMA plus 2 μg/ml of ionomycin for 6 h. Brefeldin A (10 μg/ml) was added during the last 2 h, as understood by those of skill in the art. The cells were stained with a combination of PE-labeled mAbs to IL-4 or TNF-α, FITC-labeled mAbs to IFN-γ, and APC-labeled anti-IL-10 (all from BD) using FIX and PERM kit (CALTAG), as understood by those of skill in the art.

T cell expansion and suppressive function assay. T cells were collected and resuspended in an EDTA-containing medium to dissociate the clusters, as understood by those of skill in the art. Viable cells were counted by trypan-blue exclusion of the dead cells, as understood by those of skill in the art. For suppressive function assay, naïve CD4⁺ T cells (A) and Tr1 cells generated from naïve CD4⁺ T cells by anti-CD3 mAb, anti-CD28 mAb, IL-2, Dex, and vit D3 in the presence of parental L cells (B) or OX40L-L cells (C), these three cell types and their mixtures at a 1:1 ratio were then restimulated for 5 days by culturing in the presence of 5 μg/ml anti-CD3 mAb and 1 μg/ml anti-CD28 mAb, after which time the cellular proliferation was assessed by [³H]thymidine incorporation, as understood by those of skill in the art.

In the drawings and specification, there have been disclosed embodiments of the invention and, although specific terms are employed, they are used in a generic and descriptive sense only and not for the purpose of limitation, the scope of the invention being set forth in the following claims.

We claim:

1. A method of treating cancer comprising the step of administering to a subject in need thereof a therapeutically effective amount of an antibody that modulates the activation of the OX40 receptor, wherein the immunosuppressive function of Tr1 cells and Foxp3⁺ T-reg cells is inhibited.

2. The method of claim 1 wherein the substance is an antibody agonist to an OX40-receptor.

3. The method of claim 1 wherein IL-10 production is inhibited.

4. The method of claim 1 wherein the generation of Tr1 cells from naïve or CD4⁺ cells is inhibited.

5. The method of claim 1 wherein the cancer is B cell lymphoma.

6. A method to treat B cell lymphoma comprising the step of administering to a subject in need thereof a therapeutically effective amount of OX40 ligand or other OX40 receptor agonist.

7. The method of claim 6 wherein the substance is an anti-OX40 antibody agonist.

8. A high throughput screening method for compounds that modulate the OX40 receptor comprising the steps of:
(A) transfecting T cells having the ability to produce IL-10 with an OX40-gene;
(B) culturing the transfected T cells with fibroblast cells and a substance of interest;
(C) collecting the culture supernatants; and
(D) analyzing the IL-10 content of the culture supernatants, wherein antibodies or small molecules which inhibit the binding of the OX40-ligand to the OX40 receptor are produced.
